(19) United States
(12) Patent Application Publication
(10) Pub. No.: US 2011/0245880 A1
(43) Pub. Date: Oct. 6, 2011

(54) SPINAL FIXATOR AND METHOD OF USE THEREOF

(75) Inventor: MYSLIWIEC LAWRENCE, Williamston, MI (US)

(73) Assignee: THE BOARD OF TRUSTEES OF MICHIGAN STATE UNIVERSITY, East Lansing, MI (US)

(21) Appl. No.: 13/076,865

(22) Filed: Mar. 31, 2011

Related U.S. Application Data
(60) Provisional application No. 61/319,998, filed on Apr. 1, 2010.

(51) Int. Cl. A61B 17/88; A61B 17/70

(52) U.S. Cl. 606/279; 606/246

(57) ABSTRACT

There is provided an explantable system having at least one pedicle screw, and a fixation plate. The system is a base plate including at least one aperture extending through the base plate screw retaining means sized to fit within the aperture for maintaining screw means therein. The system is designed to be removed after a predetermined period of time.

Also provided is A method of implanting the fixation device by wire guiding the assembly in place using minimally invasive surgery and fixing the assembly in place. Further, the assembly can also be removed or explanted using minimally invasive surgery.

### BACKGROUND OF THE INVENTION

1. Field of the Invention

Generally, the present invention relates to implants. More specifically, the present invention relates to spinal implants.

2. Description of the Related Art

It has been estimated that 70% of adults have had a significant episode of back pain or chronic back pain emanating from a region of the spinal column or backbone. Many people suffering chronic back pain or an injury requiring immediate intervention resort to surgical intervention to alleviate their pain.

The spinal column or back bone encloses the spinal cord and consists of 33 vertebrae superimposed upon one another in a series which provides a flexible supporting column for the trunk and head. The vertebrae cephalad (i.e., toward the head or superior) to the sacral vertebrae are separated by fibrocartilaginous intervertebral discs and are united by articular capsules and by ligaments. The uppermost seven vertebrae are referred to as the cervical vertebrae, and the next lower twelve vertebrae are referred to as the thoracic, or dorsal, vertebrae. The next lower succeeding five vertebrae below the thoracic vertebrae are referred to as the lumbar vertebrae and are designated L1-L5 in descending order. The five vertebrae below the lumbar vertebrae are referred to as the sacral vertebrae and are numbered S1-S5 in descending order. The final four vertebrae below the sacral vertebrae are referred to as the coccygeal vertebrae. In adults, the five sacral vertebrae fuse to form a single bone referred to as the sacrum, and the four rudimentary coccyx vertebrae fuse to form another bone called the coccyx or commonly the “tail bone”. The number of vertebrae is sometimes increased by an additional vertebra in one region, and sometimes one may be absent in another region.

Typical lumbar, thoracic and cervical vertebrae consist of a ventral or vertebral body and a dorsal or neural arch. In the thoracic region, the ventral body bears two costal pits for reception of the head of a rib on each side. The arch which encloses the vertebral foramen is formed of two pedicles and two lamina. A pedicle is the bony process which projects backward or anteriorly from the body of a vertebra connecting with the lamina on each side. The pedicle forms the root of the vertebral arch. The vertebral arch bears seven processes: a dorsal spinous process, two lateral transverse processes, and four articular processes (two superior and two inferior). A deep concavity, inferior vertebral notch, on the inferior border of the arch provides a passageway or spinal canal for the delicate spinal cord and nerves. The successive vertebral foramina surround the spinal cord. Articulating processes of the vertebrae extend posteriorly of the spinal canal.

The bodies of successive lumbar, thoracic and cervical vertebrae articulate with one another and are separated by intervertebral discs formed of fibrous cartilage enclosing a central mass, the nucleus pulposus that provides for cushioning and dampening of compressive forces to the spinal column. The intervertebral discs are anterior to the vertebral canal. The inferior articular processes articulate with the superior articular processes of the next succeeding vertebra in the caudal (i.e., toward the feet or inferior) direction. Several ligaments (supraspinous, interspinous, anterior and posterior longitudinal, and the ligamenta flava) hold the vertebrae in position yet permit a limited degree of movement.

The relatively large vertebral bodies located in the anterior portion of the spine and the intervertebral discs provide the majority of the weight bearing support of the vertebral column. Each vertebral body has relatively strong bone comprising the outside surface of the body and weak bone comprising the center of the vertebral body.

Various types of spinal column disorders are known and include scoliosis (abnormal lateral curvature of the spine), kyphosis (abnormal forward curvature of the spine, usually in the thoracic spine), excess lordosis (abnormal backward curvature of the spine, usually in the lumbar spine), spondylolisthesis (forward displacement of one vertebra over another, usually in the lumbar or cervical spine) and other disorders, such as ruptured or slipped discs, degenerative disc disease, fractured vertebra, and the like. Patients who suffer from such conditions usually experience extreme and debilitating pain and often neurologic deficit in nerve function.

Approximately 95% of spinal surgery involves the lower lumbar vertebrae designated as the fourth lumbar vertebra (“L4”), the fifth lumbar vertebra (“L5”), and the first sacral vertebra (“S1”). Persistent low back pain is attributed primarily to degeneration of the disc connecting L5 and S1. There are two possible mechanisms whereby intervertebral disc lesions can instigate and propagate low back pain. The first theory proposes that the intervertebral disc itself produces pain through trauma or degeneration and becomes the primary source of low back pain. Proponents of this theory advocate removal of the painful disc to relieve the low back pain.

Two extensive procedures are available to remove the disc and fuse the adjacent vertebrae together. One method is to replace the disc with bone plugs by going through the spinal canal on either side of the central nerve bundle. This method requires extensive stripping of the paraspinal musculature. More importantly, there are extensive surgical manipulations within the spinal canal itself. Although the initial proponents of this approach report 90% excellent to good results, subsequent studies have been unable to obtain acceptable outcomes and recommend adding internal fixation to improve fusion rates.

The second procedure is the anterior lumbar fusion which avoids the morbidity of posterior muscle stripping by approaching the spine through the abdomen. Surgeons experienced with this technique also report good to excellent patient results in 90% of cases performed. However, when generally used by practicing surgeons, the procedure was found to have a high failure rate of fusion. Attempts to increase the fusion rate by performing a posterior stabilization procedure have been successful, but the second incision increases the morbidity and decreases the advantages of the technique. Thus, the present surgical techniques available to remove and fuse painful lumbar discs are extensive operative procedures with potentially significant complications.

There is no single procedure that is universally accepted to surgically manage low back pain patients. Although damaged discs and vertebral bodies can be identified with sophisticated diagnostic imaging, the surgical procedures are so extensive that clinical outcomes are not consistently satisfactory. Furthermore, patients undergoing presently available fusion surgery experience uncomfortable, prolonged convalescence.

A number of devices and techniques involving implantation of spinal implants to reinforce or replace removed discs and/or anterior portions of vertebral bodies and which mechanically immobilize areas of the spine assisting in the eventual fusion of the treated adjacent vertebrae have also been employed or proposed over the years In order to overcome the disadvantages of purely surgical techniques. Such techniques have been used effectively to treat the above-described conditions and to relieve pain suffered by the patient. However, there are still disadvantages to the present fixation implants and surgical implantation techniques. The historical development of such implants is set forth in U.S. Pat. Nos. 5,505,732, 5,514,180, and 5,888,223, for example, all incorporated herein by reference.

One technique for spinal fixation includes the immobilization of the spine by the use of spine rods of many different configurations that run generally parallel to the spine. Typically, the posterior surface of the spine is isolated and bone screws are first fastened to the pedicles of the appropriate vertebrae or to the sacrum and act as anchor points for the spine rods. The bone screws are generally placed two per vertebra, one at each pedicle on either side of the spinous process. Clamp assemblies join the spine rods to the screws. The spine rods are generally bent to achieve the desired curvature of the spinal column. Wires may also be employed to stabilize rods to vertebrae. These techniques are described further in U.S. Pat. No. 5,415,661, for example, incorporated herein by reference.

These types of rod systems can be effective, but require a posterior approach and implanting screws into or clamps to each vertebra over the area to be treated. To stabilize the implanted system sufficiently, one vertebra above and one vertebra below the area to be treated are often used for implanting pedicle screws. Since the pedicles of vertebrae above the second lumbar vertebra (L2) are very small, only small bone screws can be used which sometimes do not give the needed support to stabilize the spine. These rods and screws and clamps or wires are surgically fixed to the spine from a posterior approach, and the procedure is difficult. A large bending moment is applied to such rod assemblies, and because the rods are located outside the spinal column, they depend on the holding power of the associated components that can pull out of or away from the vertebral bone.

In a variation of this technique disclosed in U.S. Pat. Nos. 4,553,273 and 4,636,217 (both described in U.S. Pat. No. 5,735,899 incorporated herein by reference, two of three vertebrae are joined by surgically obtaining access to the interior of the upper and lower vertebral bodies through excision of the middle vertebral body. In the '899 patent, these approaches are referred to as “intraosseous” approaches, although they are more properly referred to as “interosseous” approaches by virtue of the removal of the middle vertebral body. The removal is necessary to enable a lateral insertion of the implant into the space it occupied so that the opposite ends of the implant can be driven upward and downward into the upper and lower vertebral bodies. These approaches are criticized as failing to provide adequate medial-lateral and rotational support in the '899 patent. In the '889 patent, an anterior approach is made, slots are created in the upper and lower vertebrae, and rod ends are fitted into the slots and attached to the remaining vertebral bodies of the upper and lower vertebrae by laterally extending screws.

A wide variety of anterior, extraosseous fixation implants, primarily anterior plate systems, have also been proposed or surgically used. One type of anterior plate system involves a titanium plate with unicortical titanium bone screws that lock to the plate and are placed over the anterior surface of a vertebral body. Another type of anterior plate system involves the use of bicortical screws that do not lock to the plate. The bone screws have to be long enough to bite into both sides of the vertebral body to gain enough strength to obtain the needed stability. These devices are difficult to place due to the length of the screws, and damage occurs when the screws are placed improperly.

A number of disc shaped replacements or artificial disc implants and methods of insertion have been proposed as disclosed, for example, in U.S. Pat. Nos. 5,514,180 and 5,888,223, for example. A further type of disc reinforcement or augmentation implant that has been clinically employed for spinal fusion comprises a hollow cylindrical titanium cage that is externally threaded and is screwed laterally into place in a bore formed in the disc between two adjacent vertebrae. Bone grafts from cadavers or the pelvis or substances that promote bone growth are then packed into the hollow center of the cage to encourage bone growth through the cage pores to achieve fusion of the two adjacent vertebrae. Two such cage implants and the surgical tools employed to place them are disclosed in U.S. Pat. Nos. 5,505,732 and 5,700,291, for example. The cage implants and the associated surgical tools and approaches require precise drilling of a relatively large hole for each such cage laterally between two adjacent vertebral bodies and then threading a cage into each prepared hole. The large hole or holes can compromise the integrity of the vertebral bodies, and if drilled too posteriorly, can injure the spinal cord. The end plates of the vertebral bodies, which comprise very hard bone and help to give the vertebral bodies needed strength, are usually destroyed during the drilling. The cylindrical cage or cages are now harder than the remaining bone of the vertebral bodies, and the vertebral bodies tend to collapse or “telescope,” together. The telescoping causes the length of the vertebral column to shorten and can cause damage to the spinal cord and nerves that pass between the two adjacent vertebrae.

Methods and apparatus for accessing the discs and vertebrae by lateral surgical approaches are described in U.S. Pat. No. 5,976,146. The intervening muscle groups or other tissues are spread apart by a cavity forming and securing tool set disclosed in the '146 patent to enable endoscope aided, lateral access to damaged vertebrae and discs and to perform corrective surgical procedures.

A compilation of the above described surgical techniques and spinal implants and others that have been used clinically is set forth in certain chapters of the book entitled Lumbosacral and Spinopelvic Fixation, edited by Joseph Y. Margolies et al. (Lippincott-Raven Publishers, Philadelphia, 1996).

The above-described spinal implant approaches involve highly invasive surgery that laterally exposes the anterior or posterior portions of the vertebrae to be supported or fused. Extensive muscular stripping and bone preparation can be necessary. As a result, the spinal column can be further weakened and/or result in surgery induced pain syndromes. Thus, presently used surgical fixation and fusion techniques involving the lower lumbar vertebrae suffer from numerous disadvantages. It is preferable to avoid the lateral exposure to correct less severe spondylolisthesis and other spinal injuries or defects affecting the lumbar and sacral vertebrae and discs.

Accordingly, it would be desirable to provide a device that reduces the difficulties risks of the current procedures. It would also be desirable to provide a device that can be placed in a less disruptive or less invasive manner than commonly used procedures.

### SUMMARY OF THE INVENTION

According to the present invention there is provided an explantable system having at least one pedicle screw, and a fixation plate. The system is designed to be removed after a predetermined period of time.

Also provided is a method of implanting the fixation device by wire guiding the assembly in place using minimally invasive surgery and fixing the assembly in place. Further, the assembly can also be removed or explanted using minimally invasive surgery.

These and other objects, advantages, and features of the invention will be more fully understood and appreciated by reference to the description of the current embodiment and the drawings.

### BRIEF DESCRIPTION OF THE DRAWINGS

Other advantages of the present invention will be readily appreciated as the same becomes better understood by reference to the following detailed description when considered in connection with the accompanying drawings wherein

FIG. 1 is a profile view of a nut for use with an embodiment of the present invention;

FIG. 2 is a profile view of a base plate and nut of an embodiment of the present invention;

FIGS. 3A-C are top views of different sized base plates for use with an embodiment of the present invention;

FIGS. 4A-C are sides views of different sized screw maintaining mechanisms for use with an embodiment of the present invention;

FIGS. 5A-C are side views of a K-wire (FIG. 5A), calibrated probe (FIG. 5B), and calibrated cannula (FIG. 5C) for use with an embodiment of the present invention;

FIGS. 6A-C are side views of different sized pedicle screws for use with an embodiment of the present invention;

FIG. 7 is a side view of a T-handle tap device for use with an embodiment of the present invention;

FIGS. 8A and B a side view (FIG. 8A) and close up view (FIG. 8B) of a T-handle drill device used to insert polyaxial pedicles screws for use with in the present invention;

FIG. 9 is a side view of a nut driver for use with an embodiment of the present invention;

FIG. 10 is a side view of a screw driver for use with an embodiment of the present invention;

FIGS. 11A-C are side views of different sized pedicle screws for use with another embodiment of the present invention showing a tapered bone screw for pedicle-bone placement on one end, and machine threading on other end (exemplifying the hanger screw principle);

FIGS. 12A-C are sides views of different sized screw maintaining mechanisms for use with another embodiment of the present invention showing a coupler-nut that is threaded over the machined thread- portion of the screw, similar to a hanger screw;

FIG. 13 is a profile view of a nut for use with another embodiment of the present invention;

FIG. 14 is a side view of the present invention;

FIG. 15A-C are top views of different sized base plates for use with another embodiment of the present invention; and

FIG. 16 side view of the assembled system of the present invention incorporating the pedicle/hanger screws; coupler nuts; sub-cutaneous plates; and locking nuts.

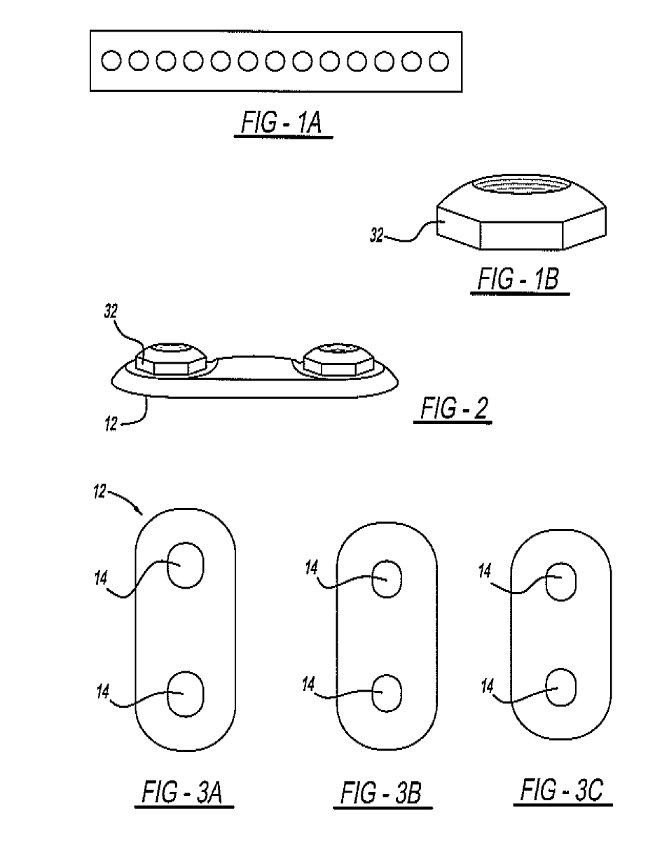

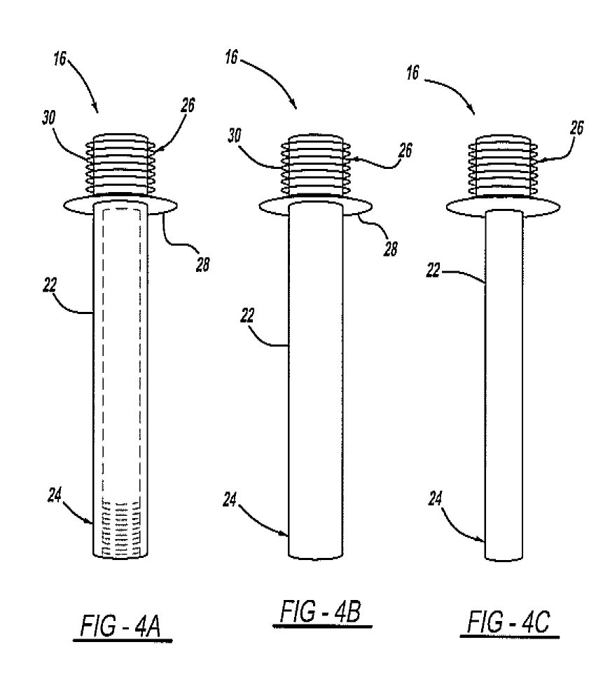

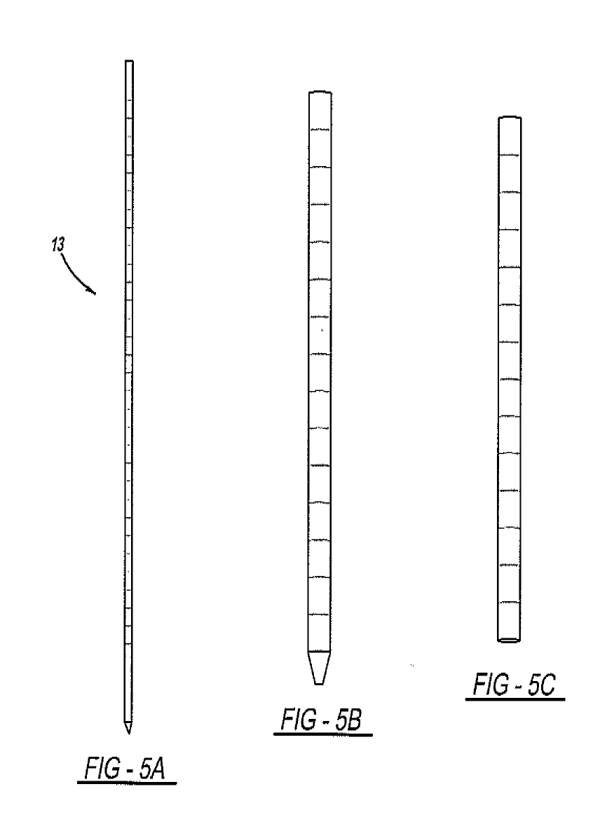

### DETAILED DESCRIPTION OF THE PREFERRED EMBODIMENTS

The present invention provides an explantable implant system that provides temporary rigid fixation of the spine in spinal fusion surgery. The system is implanted through a minimally invasive surgical approach, examples of which are well known to those of skill in the art. The system can be explanted through minimally invasive surgery after sufficient bone healing has occurred, examples of which are well known to those of skill in the art.

An explantable implant system made in accordance with the present invention is generally shown at 10 in the Figures. Primed numbers indicate like structure amongst the several embodiments. Each of the systems 10 shown can be explanted after sufficient bone fusion has occurred. As explained below in greater detail, the system enables the use of a less invasive procedure, for both the implant and explants, and limits the supra-physiologic stiffness and risk of infection. Additionally, the system enables post-operative MRI of the spine. The present invention can also be utilized in conjunction with other implants. For example, the system 10 can be utilized with fusion cages as well as other spinal fixation devices, examples of which are known to those of skill in the art.

The explantable implant system includes at least two polyaxial pedicle screws 18 and a base plate 12. The system can also include a screw retaining mechanism to maintain the pedicle screws 18 in place. The system 10 can be implanted using X-ray image technology and K-wires 13.

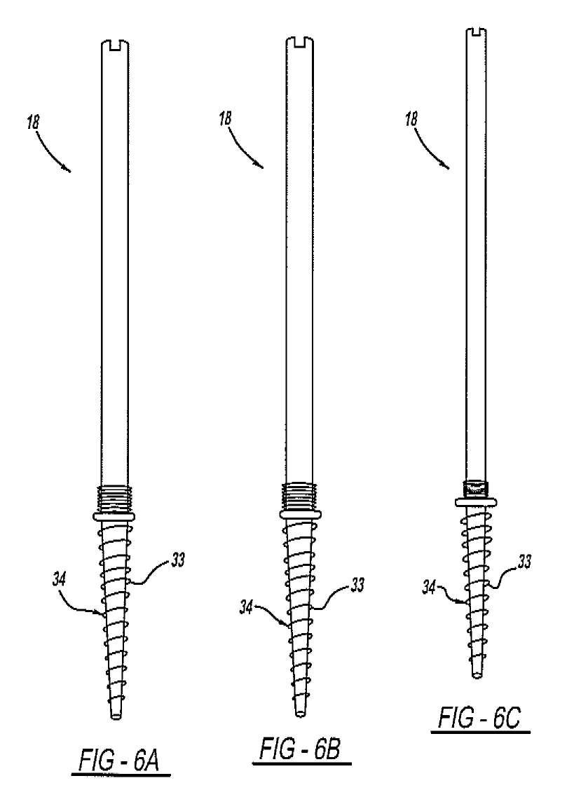

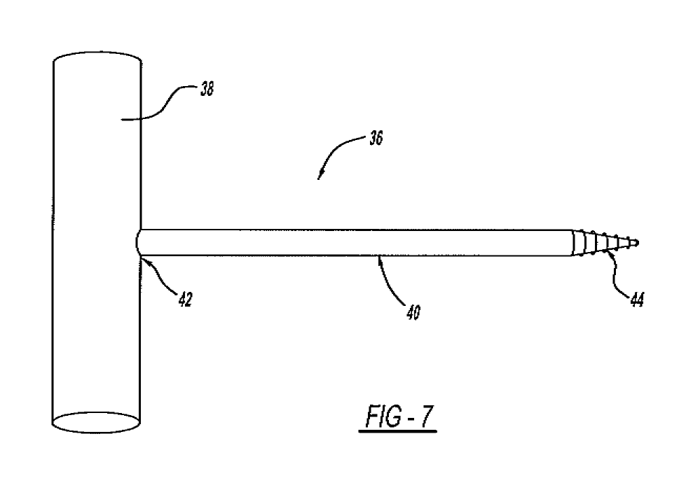

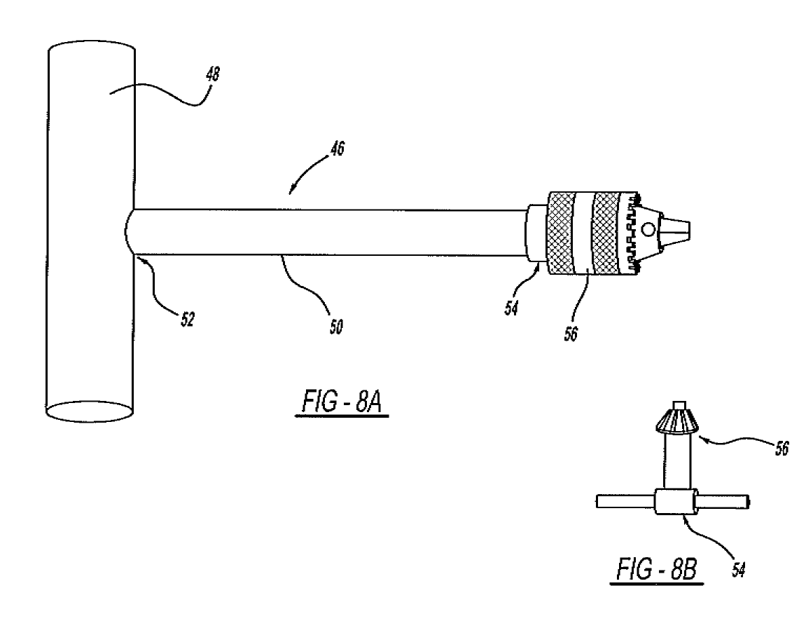

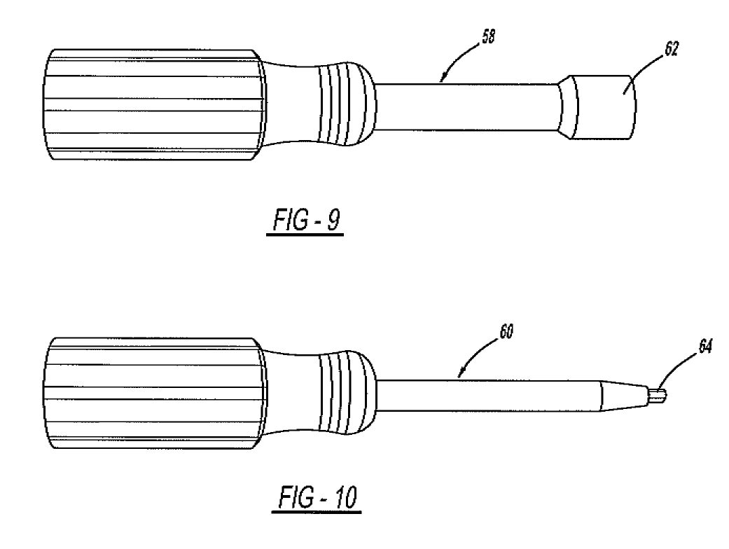

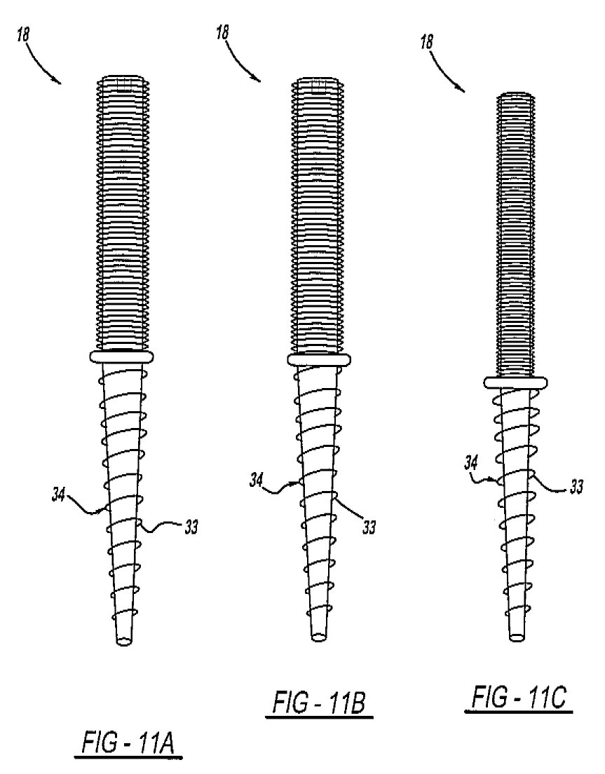

A polyaxial pedicle screw 18 (hereinafter “screw”) is a vertebral fixing device that includes a screw-type fixing anchor installed into the pedicle of a vertebra or a sacrum to immobilize a damaged spinal portion after restoring it to a normal condition, and fastening members for coupling anchors to the connecting rod. The cannulated pedicle screw is an elongate or cannulated member that can be formed by a rigid or elastic or partially flexible material having a straight posterior portion and a conical anterior portion. The ratio of straight portion to conical portion can be altered as necessary for the desired application. As shown in FIG. 6, the ratio is approximated 4:1. The cannulated member may or may not be fenestrated.

The “fixation plate” is a rigid metal or polymeric plate positioned to span bones or bone segments that require immobilization with respect to one another. The plate is fastened to the respective bones, usually with screws, so that the plate remains in contact with the bones and fixes them in a desired position. Bone plates can be useful in providing the mechanical support necessary to keep vertebral bodies in proper position and bridge a weakened or diseased area such as when a disc, vertebral body or fragment has been removed. Alternatively, subcutaneous locking plate systems that are used in the treatment of extremity fractures in orthopedic surgery. As disclosed above, alternative fixation devices can also be utilized in conjunction with the assembly without departing from the spirit of the present invention.

An “X-ray image intensifier (XRII)” as used herein, sometimes referred to as a C-Arm or fluoroscope, is a highly complex piece of equipment that uses x-rays and produces a ‘live’ image feed that is displayed on a TV screen. The device allows low intensity x-rays to be amplified, resulting in a smaller dose to the patient. The overall system consists of an x-ray source, input window, input phosphor, photocathode, vacuum and electron optics, output phosphor and output window. It allows for lower x-ray doses to be used on patients by magnifying the intensity produced in the output image, enabling the viewer to easily see the structure of the object being imaged. The use of such a device for a spinal fixation procedure is well known to those of skill in the art.

“Kirschner wires” or K-wires or pins, as used herein, are sterilized, sharpened, smooth stainless steel, or other equivalent material, pins. The K-wires are available in a variety of sizes and are used to hold bone fragments together (pin fixation) or to provide an anchor for skeletal traction. The pins are often driven into the bone through the skin (percutaneous pin fixation) using a power or hand drill. Additionally, “Denham Pins” are strong stout wires with a threaded portion in the middle. They are used for skeletal traction with the threads engaging the bone. Any of these devices can be used in conjunction with the assembly of the present invention.

Referring more specifically to the drawings, the fixation assembly 10 includes a base plate 12 having at least one aperture 14 extending therethrough for maintaining a screw retaining mechanism 16. The fixation assembly 10 utilizes at least one pedicle screw 18 for the fixation of bones, and more specifically adjacent vertebrae. The screw retaining mechanism 16 prevents the screw 18 from backing out from its fixed position within the aperture 14 of the base plate 12 and within the bone. Although there are numerous embodiments of both the fixation assembly 10 and the screw retaining mechanism 16, they all have the common characteristic of being able to cover at least a portion of the screw 18 after the screw 18 is inserted and turned into its fixed position within the base plate 12 and bone therein.

The fixation assembly 10 and the screw retaining mechanism 16 of the present invention can all be constructed of any suitable material known to those of skill in the art. Preferably, the fixation assembly 10 and the screw retaining mechanism 16 are constructed of suitable material that is compatible with uses and environments into which they are utilized. Both the fixation assembly 10 and the screw retaining mechanism 16 are constructed of metallic materials that include, but are not limited to, titanium, stainless steel, and any other metallic alloys known to those of skill in the art. Additional materials can also be utilized either alone or in combination with the metallic materials described herein. For instance, various plastics can be used. Typically though, any of the material used to construct the present invention should be very strong, non-reactive, and non-antigenic to biological systems. If the present invention is utilized outside of biological systems however, the aforementioned characteristics are not necessarily required.

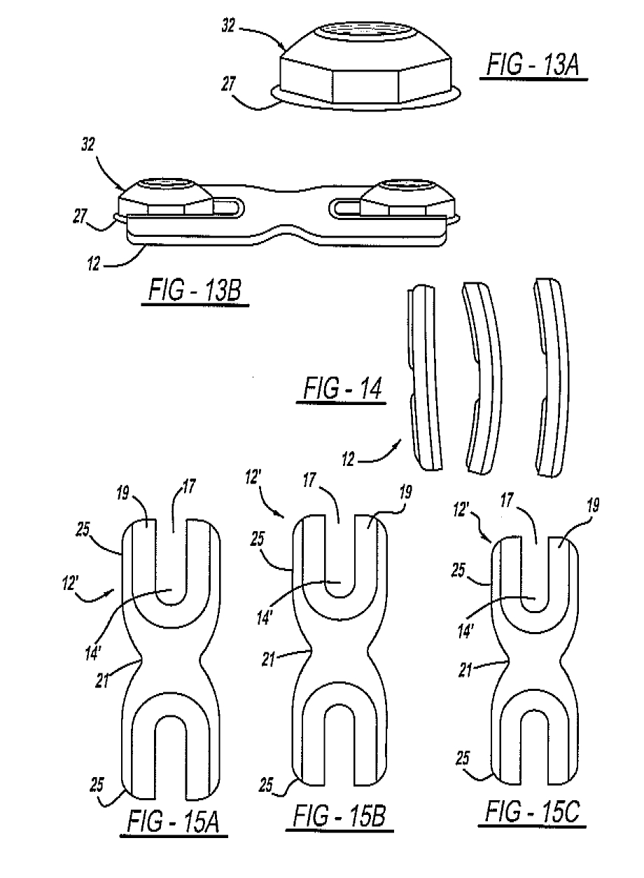

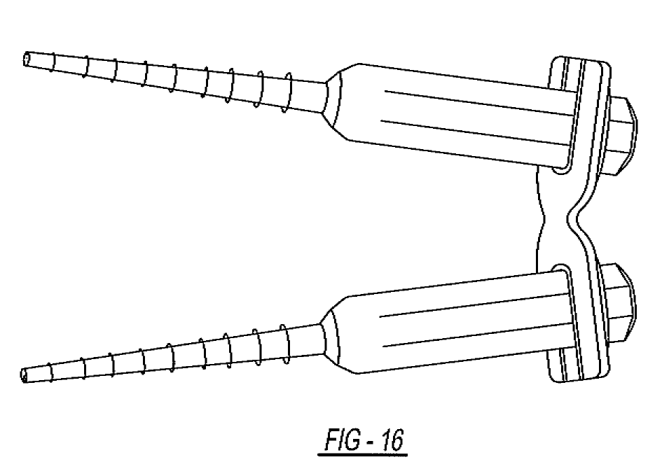

The terms “aperture” or “apertures” 14 as used herein, are meant to include, but are not limited to, any circular hole, oblong hole, slot, elongated slot, through hole, void, and any other similar opening. The aperture 14 should be large enough to accommodate at least a shaft of a screw 18. The aperture 14 is not necessarily limited to just the size of the screw 18. The aperture 14 can be larger than the screw 18, but also have a spherical seat or other similar, machined portion on the base plate 12 located therein to prevent the screw from passing completely through the aperture 14. Additionally, the aperture 14 can be an elongated slot wherein the screw 18 is capable of sliding within the slot, but is also able to accommodate the screw retaining mechanism 16 to prevent the screw 18 from backing out from its fixed position. In another embodiment (shown in FIG. 13B), the aperture 14 is a U-shaped slot 14′ having an open end 17 that allows the plate 12′ to be slid into position. The slot 14′ is sized to maintain the shaft of the screw 18 and the screw retaining mechanism 16. The aperture face 19 can either be flat, beveled, or otherwise constructed to allow for proper positioning of the screw 18 and the screw retaining mechanism 16 therein. The middle portion 21 of the plate 12 can either be the same width as the end portions 25. Alternatively, the middle portion 21 can have a smaller width than the end portions 25. This embodiment enables the end portions 25 to be twisted relative to one another to contour the plate 12′ to the needed shape.

The base plate 12 varies in size and shape. The base plate 12 can be curved, as shown in FIG. 14, to match the curvature of the spinal column. Alternatively, the base plate 12 can be completely flat, beveled, or include straight sides. According to the location and use, the size and shape of the base plate 12 can vary. The base plate 12 also includes at least one aperture 14 extending through an upper surface 13 of the base plate 12 and through a lower surface 15. The upper surface 13 is the location of the initial insertion of the screws 18 and is not touching any bone surface thereon. The lower surface 15 is closest to the spinal column and typically touches the surface of the bones to which the base plate 12 is affixed. The number of apertures 14 located on the base plate 12 vary according to design, location and severity of fixation desired. To one skilled in the art, variations can be made to provide for numerous other configurations. Thus, the present invention is not limited to those embodiments described herein. Of note, the Figures illustrate a fixation assembly 10 for cervical areas and are curved in both the longitudinal and transfer planes. Other plates of the present invention however, can utilize the screw retaining mechanism 16, while being flat or curved as is required or designed by those skilled in the art.

As previously mentioned, the apertures 14 vary in size according to the desired design of the base plate 12. For instance, the aperture 14 can be an elongated slot that allows for a substantial margin of adjustment and proper location of the screw 18 and fixation assembly 10. As a result, the screws 18 can be made to slide freely within the slots along with the screw retaining mechanism 16. The spacing and orientation of the apertures 14 within the fixation assembly 10 can be designed and selected so as to achieve a desired load sharing arrangement between the screws 18 disposed in the various combinations of apertures 14 described herein. That is, the fixation assembly 10 can be tailored to a specific application such that the load carried by each screw 18 can be distributed in a desired manner, including load shifting after the fixation assembly 10 has been affixed to the bones. The fixation assembly 10 can accommodate the dynamic environment into which it is utilized.

The apertures 14 of the fixation assembly 10 are specifically designed and machined into the base plate 12 therein to allow for the insertion of a screw retaining mechanism 16 over the screw 18 that is extended through the aperture 14 of the fixation assembly 10. The apertures 14 are machined according to desired designs and according to the type of screw retaining mechanism 16 that is utilized. Therefore, if a circular or disc shaped screw retaining mechanism 16 is utilized, then the appropriate aperture 14 is machined into the fixation assembly 10 in order to accommodate that particular type of screw retaining mechanism 16. Alternatively, the aperture 14 can be designed for use with a specially designed insert for accommodating the screw retaining mechanism 16. Such an insert for the screw retaining mechanism 16 is described below.

The screw retaining mechanism 16 can be any structure capable of preventing the screw 18 from backing out from its fixed positioned. Basically, the screw retaining mechanism 16 partially covers at least a portion of the screw 18 to prevent the screw 18 from backing out from its fixed position within the bone and from within the aperture 14 extending therethrough within the base plate 12 of the fixation assembly 10. Various embodiments of the screw retaining mechanism 16 are described herein.

In one embodiment of the present invention, the screw retaining mechanism 16 is a coupling bolt 22. The coupling bolt 22 includes an elongate hollow interior that surrounds the screw 18. The insertion end 24 of the coupling bolt 22 can be either flat or can include a threaded interior in mating alignment with the screw 18. The opposite end 26 of the coupling bolt 22 includes a collar 28 that aligns with the aperture 14 of the base plate 12 for preventing the screw 18 from backing out. Further, the opposite end 26 includes external threads 30. A locking nut 32 can be threaded about the threads 30 of the opposite end 26 of the coupling bolt 22 for preventing the screw 18 from backing out. Additionally, the locking nut 32 can be used in conjunction with an interference nut 27. The interference nut 27 is preferably a flat disc formed of a material known to those of skill in the art. One example of such a material is stainless steel. The interference nut 27 helps to maintain the screw 18 in position and works in conjunction with the locking nut 32. Alternative screw maintaining mechanisms can be utilized without departing from the spirit of the present invention.

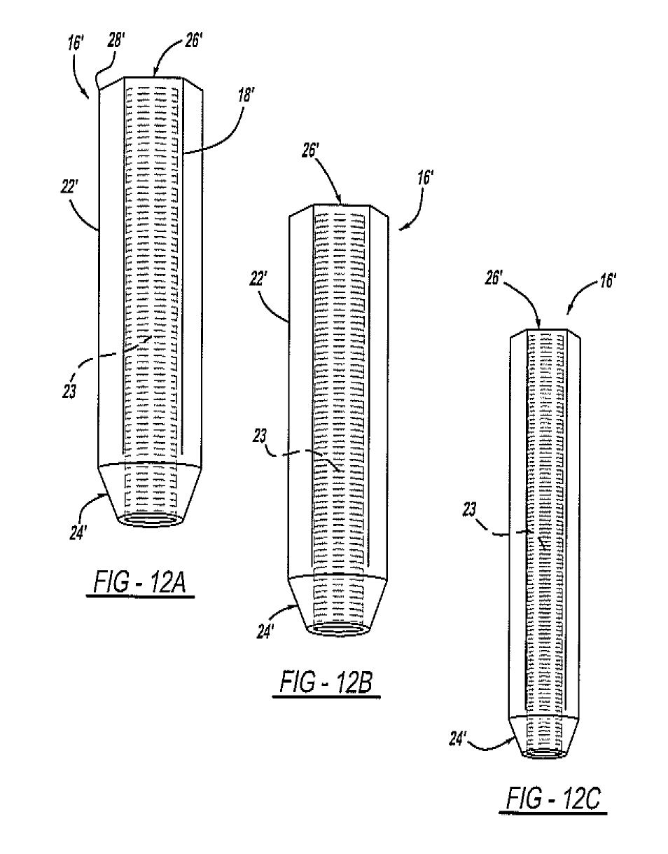

In another embodiment of the present invention, the screw retaining mechanism 16′ is a coupler nut or sleeve 22′. The coupler nut 22′ includes an elongate hollow interior 23 that surrounds the screw 18. The insertion end 24′ of the coupler nut 22′ is tapered as shown in FIGS. 12A-12C. The interior 23 of the coupler nut 22′ can include a threaded interior in mating alignment with the screw 18 or a smooth surface. The opposite end 26′ of the coupler nut 22′ includes a collar 28′ that aligns with the aperture 14 of the base plate 12 for preventing the screw 18 from backing out.

The screw 18 includes a cannula (not shown) to enable the screw to be placed about a K-wire 13 during implantation. The insertion/conical end 34 of the screw 18 includes threads 33. The size and shape of the threads can be altered to fit the desired use. For example, the spacing between threads 33 can be changed or the shape of the actual threads 33 themselves can also be changed. The distal/straight end 35 can be either smooth or threaded. Alternatively, the entire screw 18 can be threaded.

Various devices can be used during the implantation procedure. Examples of many devices are well known to those of skill in the art. For example, the T-handle tap 36 is a tapping device consisting of a cylindrical handle 38 having a long, thin cylindrical arm 40 extending from the center 42 of the handle 38. The end opposite the handle 44 is tapered and can be either smooth or threaded depending upon the intended use. The T-handle tap 36 is used in tapping cannulas and other instruments in place during the procedure. The T-handle tap 36 can be made of either a single piece of material or multiple materials. The tapered arm 40 is formed of a metal or suitable polymer that is non-reactive when used during medical procedures, examples of which are well known to those of skill in the art.

A T-handle drill 46 is a drill consisting of a cylindrical handle 48 having a cylindrical arm 50 extending from the center 52 of the handle 48. The end opposite the handle 54 includes a drilling device 56 for drilling holes for placement of the screws 18.

A nut driver 58 and hex head screw driver 60 can also be used. The head of each, 62 and 64 respectively, include heads designed to tighten screws of screw maintaining mechanisms. The heads 62, 64 can be formed to tighten any shaped screw or screw maintaining mechanism.

The devices described herein may be formed of a material that provides sufficient column strength including, but not limited to, suitable polymers, e.g. PEAK, titanium, steel, carbon fiber, and other similar materials known to those of skill in the art.

The system of the present invention can be used with any spinal fixation device. For example, the system can be used in conjunction with fusion cages, bone-grafts, or other similar procedures known to those of skill in the art.

In use, the system is beneficial because it enables the unobstructed use of spinal MRI's post-operatively, it avoids long-term implant reactions and problems, it eliminates the problems of supra-physiologic stiffness associated with permanent rigid implants. This approach is based on the principles of external fixation and subcutaneous locking plate systems that are used in the treatment of extremity fractures in orthopedic surgery.

More specifically, the fixator device is placed subcutaneously and thus only requires two small stab incisions in the skin. Cannulated fixator bolts with pedicle screws enable wire-guided placement of the vertebral pedicle. The use of a locking sleeve over the fixator bolt further solidifies the fixation within the traditionally less stable trabecular bone of the pedicle and vertebral body. Additionally, the locking sleeve enables a unilateral placement option. The subcutaneous placement reduces the risk of infection.

The percutaneous technique used to gain access to the intervetebral disc is well known to those of skill in the art. Generally, this technique can be used to place a guide-wire and cannula under C-arm fluoroscopic guidance. A specially developed anchoring cannula is then placed over the cannulated guide, and tamped into place between the end plate of the two adjacent vertebrae to be fused. The annulas of the intervertebratedal disc is otherwise preserved to protect all the surrounding anatomic structures including the spinal cord, aorta, and vena cava.

The preparation of the fusion cavity begins with a nucleotomy of the disc using a standard Kambin nucleotomy ronguer. Next, various sized semaphore endplate gouges, which open and close by a principle similar to pituitary ronguer, are used to penetrate the thick inter-vertebral endplate of the adjacent vertebrae. Finally, the carrot peeler reamer is inserted through the cannula and centered within the inter-space. The reamer handle is applied to the instrument, and the instrument is rotated like a drill, while gradually expanding the arc of the disposable spring reamer-blade until it is in the fully expanded position and can be freely rotated inside the newly created fusion chamber.

Bone graft material is extracted from and then packed into the cyst using a special augur bit applied to the end of the arthroscopic shaver. Using the augur turning in the reverse direction, autogenous bone created by gouging out and reaming of the inter-vertebral fusion cavity is harvested from the site. The bone graft is then mixed with artificial bone graft. The mix is inserted into the fusion cavity using the augur turning in the forward direction. After the cavity is packed with bone, the augur is removed. The hole-defect in the annulus is then plugged shut with a cylinder of osteoconductive scaffolding.

The fixator system can then be implanted. The system is implanted by marking the exterior skin to indicate the points for inserting the two guide wires. A spine needle is inserted at the insertion point and is guided into place using the C-arm. The needle is inserted until it rests at the 9 o′clock position of the pedicle. The stylet is then removed and the needle is tapped gently in place with a mallet or other similar device to fix the needle in the superficial cortex. The needle trocar is then removed and a K-wire is inserted through the cannula of the spinal needle using C-arm guidance. The K-wire is driven a sufficient distance into the pedicle to ensure placement, for example about ½ inches into the pedicle. The needle can then be disengaged from the pedicle and removed, maintaining the K-wire in place. The same procedure can be used to insert additional K-wires. Once all of the K-wires are in place, a wire driver or other similar device can be used to insert the K-wires to the appropriate depth for pedicle screw fixation.

An incision is then made between the K-wires and the fat is retracted. A calibrated cannulated probe is then placed about each K-wire until it impacts impacts the vertebral cortex. A pedicle screw is selected based on measurements taken by the calibrated cannulated probe.

A screw placement cannula is placed about the cannulated probe and the probe is then removed. After removal, a cannulated tap is inserted over the fixed K-wire through the operating cannula to start the hole for placement of the pedicle screw. The tap is then removed and the pedicle screw is inserted in its place. Once the screw threads sufficiently engage the pedicle the K-wire is removed.

The chuck of the T-handle drill is tightened over the end of the pedicle screw and the screw is tightened in place. The drill is then removed and screw placement is carefully monitored. These steps are then repeated for each pedicle screw.

Rod cutters are then used to cut the ends of the screws to be level with the fascia. Then, screw retaining mechanisms are then placed about the screws and a tightening device is used to tighten and connect the screw maintaining mechanisms in place.

A base plate is then placed over each of the screws and screw maintaining mechanisms engage the apertures of the base plate. Finally, a nut or other similar device is used to secure the base plate in position.

The system can be removed between 3 and 6 months post-implantation, by removing the nut and base plate device and then removing the screw retaining mechanisms and screws. The treated area should have sufficient stabilization and bone growth as to no longer need the stability provided by the base plate and screw mechanism.

The invention has been described in an illustrative manner, and it is to be understood that the terminology, which has been used herein, is intended to be in the nature of words of description rather than of limitation.

Obviously, many modifications and variations of the present invention are possible in light of the above teachings. It is, therefore, to be understood that within the scope of the described invention, the invention can be practiced otherwise than as specifically described.

What is claimed is:

1. A removable fixation assembly comprising:
a base plate including at least one aperture extending through said base plate
screw retaining means sized to fit within said aperture for maintaining screw means therein.

2. The assembly according to claim 1, wherein said aperture is an elongated slot.

3. The assembly according to claim 1, wherein said screw means is a pedicle screw.

4. The assembly according to claim 3, wherein said screw includes an essentially straight end and an opposite conical end.

5. The assembly according to claim 4, wherein said straight end is smooth and said conical end is threaded.

6. The assembly according to claim 1, wherein said screw retaining means is a coupling bolt.

7. The assembly according to claim 6, wherein said coupling bolt is a hollow cylinder having a smooth interior and a threaded interior.

8. The assembly according to claim 7, wherein said coupling bolt further includes screw locking means that aligns with said aperture of said base plate for preventing said screw means from backing out.

9. The assembly according to claim 8 wherein said screw locking means is a collar having external threads and a locking nut for preventing the screw from backing out.

10. The assembly according to claim 1 for use in conjunction with another spinal assembly.

11. A method of implanting the fixation device according to claim 1, by
wire guiding the assembly in place using minimally invasive surgery; and
fixing the assembly in place.

12. The method according to claim 11, further including removing the assembly using minimally invasive surgery.
